(19) United States
(12) Patent Application Publication
(10) Pub. No.: US 2005/0130961 A1
(43) Pub. Date: Jun. 16, 2005

(54) Muscarinic M1 receptor agonists for pain management

(76) Inventors: Robert E. Davis, San Diego, CA (US); Kimberly Vanover, San Diego, CA (US); Mario Rodriguez, Lihue, HI (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/809,975

(22) Filed: Mar. 26, 2004

Related U.S. Application Data
(60) Provisional application No. 60/459,045, filed on Mar. 28, 2003.

(51) Int. Cl. A61K 31/538; A61K 31/454

(52) U.S. Cl. 514/230.5; 514/321

(57) ABSTRACT

Disclosed herein are compounds and methods for treating chronic neuropathic pain. It has been discovered that compounds that selectively interact with a muscarinic receptor subtype are effective in treating neuropathic pain. Specifically, compounds that selectively interact with the M1 muscarinic receptor subtype may be used.

### RELATED APPLICATIONS

This application claims priority to U.S. Provisional Application No. 60/459,045, entitled “Muscarinic M1 Receptor Agonists for Pain Management,” filed on Mar. 28, 2003, which is hereby incorporated by reference in its entirety.

### BACKGROUND OF THE INVENTION

1. Field of the Invention

The present invention relates to neuropathic pain. More specifically, the present invention relates to the treatment of neuropathic pain by selectively interacting with muscarinic receptor subtypes.

2. Description of the Related Art

In many patients, damage to sensory nerves is accompanied by varying degrees of pain. The experience can range from mild increased sensitivity to touch or temperature to excruciating pain. This kind of pain is termed neuropathic pain because it is thought to involve an alteration in nervous system function or a reorganization of nervous system structure. Neuropathic pain is extremely difficult to manage clinically, is usually chronic, and fails to respond to standard analgesic interventions.

Approximately 1.5% of the US population may suffer from neuropathic pain of one kind or another. This population is larger if one includes the many forms of back pain that are neurogenic in origin. Thus, neuropathic pain can be associated with nerve damage caused by trauma, by diseases such as diabetes, herpes zoster (shingles), irritable bowel syndrome, late-stage cancer, or by chemical injury (for example, as an untoward consequence of drug therapies including the antiviral drugs).

Importantly, drugs that are effective in treating inflammatory and acute pain usually are not effective in treating neuropathic pain (such as opiates and nonsteroidal anti-inflammatory agents). Conversely, compounds that alleviate neuropathic pain may not be effective in treating acute pain (for example, gapapentin, tricylic antidepressants). The currently available treatments for neuropathic pain are not expressly designed to treat these kinds of pain and therefore, not surprisingly these drugs are not highly efficacious nor do these drugs work in all patients. Thus, there is pressing need for more effective and more tolerated treatments for neuropathic pain.

One class of molecules that shows promise in managing neuropathic pain are those molecules that interact directly or indirectly with muscarinic receptors. For example, blockade of acetylcholinesterase (ACHE-I) activity elevates acetylcholine levels by preventing its degradation and secondarily leads to the simultaneous activation of all cholinergic receptors.

In humans, drugs that inhibit cholinesterase activity are effective analgesic agents. For example, the ACHE-I physostigmine causes a short acting analgesia in surgical patients when administered postoperatively. Intrathecal administration of another chemically-related ACHE-I, neostigmine, relieves acute postoperative pain, chronic neuropathic pain and potentiates the analgesic activity of intrathecally administered opiates. Of the different cholinergic receptors, both muscarinic and nicotinic receptors have been suggested to mediate the antinociceptive and allodynic response of cholinesterase inhibitors. However, the antiallodynic effects of physostigmine were blocked by muscarinic receptor antagonists but not by nicotinic receptor antagonists, suggesting that the effects of cholinesterase inhibition on this form of pain are mediated through muscarinic and not nicotinic receptor activation.

Direct acting muscarinic receptor agonists also are antinociceptive in a variety of animal models of acute pain (Bartolini et al., 1992; Brodie and Proudfit, 1984; Capone et al., 1999; Hartvig et al., 1989; Pedigo et al, 1975; Przewlocka et al., 1999; Shannon et al., 1997; Sheardown et al., 1997). These effects can be blocked by muscarinic antagonists (Bartolini et al., 1992; Hwang et al., 1999; Naguib and Yaksh, 1997; Sheardown et al. 1997). These data further support the role for muscarinic receptor activation in the control of acute pain states.

Few studies have examined the role of muscarinic receptor activation in chronic or neuropathic pain states. In these studies, the direct and indirect elevation of cholinergic tone was shown to ameliorate tactile allodynia after intrathecal administration in a spinal ligation model of neuropathic pain in rats and these effects again were reversed by muscarinic antagonists (Hwang et al., 1999; Lee et al, 2002). Thus, direct or indirect activation of muscarinic receptors has been shown to elicit both acute analgesic activity and to ameliorate neuropathic pain. Muscarinic agonists and ACHE-Is are not widely used clinically owing to their propensity to induced a plethora of adverse events when administered to humans. The undesirable side-effects include excessive salivation and sweating, enhanced gastrointestinal motility, and bradycardia among other adverse events. These side-effects are associated with the ubiquitous expression of the muscarinic family of receptors throughout the body.

With the discovery of 5 genetically unique muscarinic receptors, M(1)-M(5), with differential distributions in the body in the mid-1980s, it became possible to conceive of designing molecules that selectively interact with one of these receptor subtypes and not the others. It was thought that the design of selective molecules would permit modulation, for example, of muscarinic receptors controlling central nervous function without also activating muscarinic receptors controlling cardiac, gastrointestinal or glandular functions. Despite enormous effort, no drugs with this desired selectivity have been developed resulting principally from the structural similarity of important activation regions of these 5 receptor subtypes.

Also, it is not known which of the 5 muscarinic receptor subtypes mediate the effects of muscarinic compounds on various pain states. Indeed, it is possible that activation of more than one muscarinic receptor subtype may be involved in pain control or that activation of different muscarinic receptor subtypes may mediate different forms of pain. For example, the M(2) receptor is highly expressed in the dorsal root ganglion in the small-medium type neurons, in the dorsal horn of the spinal cord and the thalamus, suggesting that activation of M(2) receptors may participate in the modulation of the transduction of noxious stimuli from the periphery through the spinal cord to the brain. This hypothesis was confirmed by the finding that deletion of the M(2) receptors in mice reduces the acute antinociceptive activity of mucarinic agonists. Additionally, based on deletions of other muscarinic receptor subtypes in mice, only the M(2), and perhaps to a lesser extent M(4), receptors appear to contribute the acute analgesic activity of muscarinic agonists. Others have reached a similar conclusion: “These data provide unambiguous evidence that muscarinic analgesia is exclusively mediated by a combination of M(2) and M(4) muscarinic receptors at both spinal and supraspinal sites” (Duttaroy A, et al, 2002). Further, still others have noted: “However, activity at the M(1) receptor subtype is not a requirement for antinociceptive activity” (Sheardown, et al, 1997).

Notwithstanding these data, the therapeutic utility of a compound acting directly at M(2) receptors is limited. This is because the M(2) receptor also is highly expressed in the heart and the GI tract, suggesting that this receptor also mediates the gastrointestinal distress and cardiovascular side effects of muscarinic receptors. Again, this suggestion was confirmed in mice with deletions of the M(2) receptor. Thus, agents that directly or indirectly activate M(2) muscarinic receptors might not be useful in even treating acute pain due to unwanted and potentially dangerous side-effects.

A similar scientific compendium is not available for neuropathic pain. The precise muscarinic receptor subtype mediating the activity of direct and indirect muscarinic agonists in neuropathic pain states clearly is not known. There is a strong medical need to determine the muscarinic receptor subtype(s) that are involved in ameliorating neuropathic pain and to develop drugs that selectively activate these receptors.

### SUMMARY OF THE INVENTION

Disclosed herein is a method for treating neuropathic pain comprising identifying a subject in need of such treatment and providing the subject with an effective amount of at least one compound that selectively activates the M(1) receptor subtype, whereby one or more symptoms of the neuropathic pain are reduced. In some embodiments the subject presents hyperalgesia. In some embodiments, the subject presents allodynia. In some embodiments, the neuropathic pain is associated with diabetes, viral infection, irritable bowel syndrome, amputation, cancer, or chemical injury. In some embodiments the compound that selectively activates the M(1) receptor subtype does not alleviate acute pain. In some embodiments, the compound is selected from the group consisting of the compounds of Formulas VII, VIII, and IX:

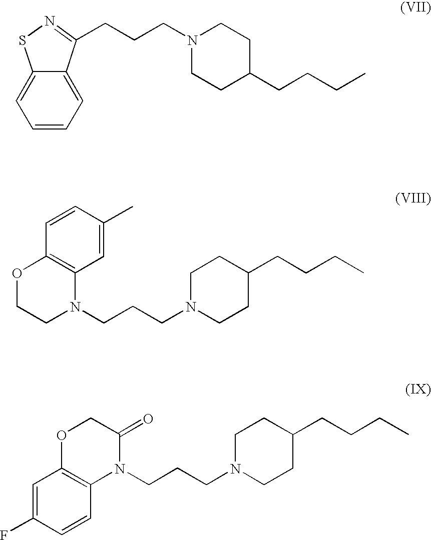

Also disclosed herein is a method of identifying a compound that alleviates hyperalgesia or allodynia in a subject, comprising providing the subject with at least one muscarinic receptor test compound and determining if the at least one test compound reduces hyperalgesia or allodynia in the subject. In some embodiments the at least one test compound is selective for the M(1) or M(4) but not M(2) or M(3) receptor. In some embodiments the at least one test compound is selective for the M(1) receptor. In some embodiments the hyperalgesia is thermal hyperalgesia. In some embodiments the allodynia is tactile allodynia.

Also disclosed herein is a pharmaceutical composition comprising an effective amount of at least one compound that selectively activates the M(1) receptor subtype in an amount effective to reduce one or more symptoms of neuropathic pain. In some embodiments the compound is selected from the group consisting of the compounds of Formulas VII, VIII, and IX.

### BRIEF DESCRIPTION OF THE DRAWINGS

FIG. 1 shows chemical structures of examples of the compound of Formula (VI).

FIG. 2 shows the effect of treatment with the compound of Formula IX on tactile sensitivity after partial sciatic ligation.

FIG. 3 shows the effect of administering the compound of Formula IX i.c.v. on tactile sensitivity after partial sciatic ligation.

### DETAILED DESCRIPTION OF THE PREFERRED EMBODIMENTS

Compounds have been developed with unprecedented selectivity for the M(1) receptor relative to other muscarinic receptor subtypes (Spalding T A, Trotter C, Skjaerbaek N, Messier T L, Currier E A, Burstein E S, Li D, Hacksell U, Brann M R. Discovery of an ectopic activation site on the M(1) muscarinic receptor. Mol. Pharmacol, 61(6):1297-302, 2002; U.S. application Ser. No. 10/262,517 (publication number 20030100545), entitled, “Benzimidazolidinone Derivatives as Muscarinic Agents”; U.S. Pat. No. 6,627,645, entitled, “Muscarinic Agonists”; U.S. Pat. No. 6,528,529, entitled, “Compounds with Activity on Muscarinic Receptors”; U.S. application Ser. No. 10/338,937 (publication number 20030144285), entitled, “Compounds with Activity on Muscarinic Receptors”; U.S. application Ser. No. 10/329,455 (publication number 20030176418), entitled, “Tetrahydroisoquinoline Analogues as Muscarinic Agonists”; and U.S. Provisional No. 60/432,692, entitled, “Piperidinyl Dimers as Muscarinic Agents”; all of which are hereby incorporated by reference in their entirety.

Compounds with relative selectivity for the M(1) muscarinic receptor have been discovered to be very effective in ameliorating thermal hyperalgesia and tactile allodynia in rodent models of neuropathic pain when administered systemically. Because these compounds also do not activate other muscarinic receptor subtypes, these M(1) agonists do not elicit the undesirable and life-threatening actions of previous nonselective muscarinic agonists. M(1) selective agonists, therefore, are particularly attractive as therapies for treating chronic neuropathic pain. Conversely, unlike nonselective muscarinic agonists that interact with M(2) and all other muscarinic receptor subtypes, these M(1) selective agonist are not effective in reducing acute pain. Thus, selective M(1) agonists have a particularly attractive profile in rodents. They block neuropathic pain but do not alter response to other forms of pain. In chronic use, these agents should allow patients to respond normally to acute pain while at the same time blocking chronic neuropathic pain.

As used herein, the term “selective” is defined as a property of a compound whereby an amount of the compound sufficient to effect a desired response from a particular receptor type, subtype, class or subclass with significantly less or substantially little or no effect upon the activity of other receptor types. For example, a selective compound may have at least a 10-fold greater effect on activity of the desired receptor than on other receptor types. In some cases, a selective compound may have at least a 20-fold greater effect on activity of the desired receptor than on other receptor types, or at least a 50-fold greater effect, or at least a 100-fold greater effect, or at least a 1000-fold greater effect, or at least a 10,000-fold greater effect, or at least a 100,000-fold greater effect, or more than a 100,000-fold greater effect.

The site of action of M(1) agonist effects on neuropathic pain remain to be elucidated. Yet, the neuropathic pain relieving effects of M(1) selective agonists have been shown to be blocked by the central nervous system penetrating muscarinic antagonist scopolamine hydrochloride but not by the mainly peripheral-acting muscarinic antagonist methylscopolamine hydrochloride. This suggests that the neuropathic pain relieving effects of M(1) selective muscarinic agonists are mediated through action in the central nervous system. Further, these M(1) selective agonists are not effective in alleviating neuropathic pain when administered intrathecally into the spinal cord but are effective alleviating this form of pain when administered intracerebroventricularly. This suggests that the neuropathic pain relieving effects of M(1) receptor activation are mediated by supraspinal and not necessarily spinal sites of action.

Compounds that interact with the M(1) receptor subtype possess heretofore unappreciated analgesic activity and are effective treatments for neuropathic pain. These observations have practical applications that support the use of M(1) agonists in the treatment of neuropathic pain caused by trauma, by diseases such as diabetes, herpes zoster (shingles), irritable bowel syndrome or late-stage cancer, or by chemical injury (for example, as an untoward consequence of drug therapies including the antiviral drugs).

Thus, in some embodiments of the present invention, neuropathic pain in an organism is treated by contacting a subject with a pharmacologically active dose of a compound that interacts with the M(1) receptor subtype for the purpose of controlling pain without also causing unwanted and utility limiting side-effects.

In some embodiments, the compounds for use in the present invention selectively interacts with the M(1) receptor subtype.

In some embodiments, the compounds for use in the present invention are described in U.S. patent application Ser. No. 10/262,517 (publication number 20030100545), the disclosure of which is hereby incorporated by reference its entirety, and have the structure of Formula (I): 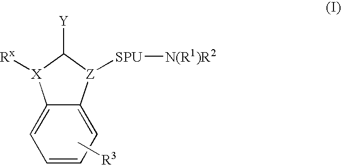
wherein
X is selected from the group consisting of C, O, N and S;
Z is selected from the group consisting of CH and N;
Y is selected from the group consisting of ═O, ═N and ═S or tautomers thereof, such as Y-alkylated tautomers;
SPU is a spacer unit providing a distance d between Z and N wherein —SPU— is a biradical selected from the group consisting of —(CR⁶R⁷)ₙ-A- and —C₃₋₈-cycloalkyl-, wherein n is in the range 1 to 5, such as 1, 2, 3, 4, or 5 and A is absent or an optionally substituted —C₃₋₈-cycloalkyl;
N together with R¹ and R² form a heterocyclic ring wherein said heterocyclic ring is selected from the group consisting of perhydroazocine, perhydroazepine, piperidine, pyrrolidine, azetidine, aziridine and 8-azabicyclo[3.2.1]octane and wherein the heterocyclic ring is substituted with one or more substituents R⁴ selected from the group consisting of hydroxy, halogen, C₁₋₈-alkyl, C₃₋₈-cycloalkyl, C₁₋₈-alkoxy, C₁₋₈-alkylcarbonyl, C₁₋₈-alkylidene, C₂₋₈-alkenyl, C₂₋₈-alkynyl, C₁₋₆-alkyloxyimino, and C₁₋₆-alkyloxyamino each of which may be optionally substituted with a substituent R⁵ and wherein at least one of said substituents R⁴ is R^{4′} selected from the group consisting of C₁₋₈-alkyl, C₃₋₈-cycloalkyl, C₁₋₈-alkoxy, C₁₋₈-alkylcarbonyl, C₁₋₈-alkylidenec C₁₋₈-alkyloxyimino, and C₁₋₈-alkyloxyamino each of which may be optionally substituted with a substituent R⁵;
R⁵ is selected from the group consisting of hydrogen, halogen, hydroxy, C₁₋₈-alkyl, C₁₋₈-alkoxy, C₃₋₈-cycloalkyl, C₃₋₈-heterocyclyl, C₁₋₈-alkylcarbonyl, C₁₋₈-alkylidene, C₂₋₈-alkenyl and C₂₋₈-alkynyl;
R^{X} may be absent or selected from the group consisting of hydrogen, optionally substituted C₁₋₈-alkyl, optionally substituted C₃₋₈-cycloalkyl, optionally substituted C₂₋₈-alkenyl, optionally substituted C₂₋₈-alkynyl, optionally substituted aryl, optionally substituted heteroaryl CH₂—N(R⁵)(R⁵), CH₂—OR⁵, CH₂—SR⁵, CH₂—O—C(═O)R⁵, CH₂—O—C(═S)R⁵;
R³ may be present 0-4 times and selected from the group consisting of halogen, hydroxy, optionally substituted C₁₋₈-alkyl, C₁₋₈-alkoxy, optionally substituted C₁₋₈-alkylidene, optionally substituted C₂₋₈-alkenyl, optionally substituted C₂₋₈-alkynyl optionally substituted aryl, optionally substituted heteroaryl, optionally substituted C₃₋₈-cycloalkyl, optionally substituted C₃₋₈-heterocyclyl, and optionally substituted C₁₋₈-alkylcarbonyl; and
each R⁶ and each R⁷ is independently selected from the group consisting of hydrogen, halogen, hydroxy, optionally substituted C₁₋₈-alkyl, C₁₋₈-alkoxy, optionally substituted C₁₋₈-alkylidene, optionally substituted C₂₋₈-alkenyl, optionally substituted C₂₋₈-alkynyl optionally substituted aryl, optionally substituted heteroaryl, optionally substituted C₃₋₈-cycloalkyl, optionally substituted C₃₋₈-heterocyclyl, and optionally substituted C₁₋₈-alkylcarbonyl.

In some embodiments, the compounds for use in the present invention are described in U.S. Pat. No. 6,627,645, the disclosure of which is hereby incorporated by reference and its entirety, and have the structure of Formula (II): 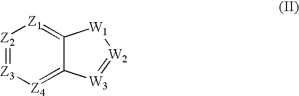
wherein:
Z₁, is CR₁ or N, Z₂ is CR₂ or N, Z₃ is CR₃ or N, and Z₄ is CR₄ or N, where no more than two of Z₁, Z₂, Z₃ and Z₄ are N;
W₁ is O, S, or NR₅, one of W₂ and W₃ is N or CR₆, and the other of W₂ and W₃ is CG; W₁ is NG, W₂ is CR₅ or N, and W₃ is CR₆ or N; or W₁ and W₃ are N, and W₂ is NG;
G is of formula (III): 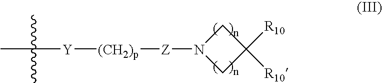
Y is O, S, CHOH, —NHC(O)—, —C(O)NH—, —C(O)—, —C(O)—, —OC(O)—, —(O)CO—, —NR₇—, —CH═N—, or absent;
p is 1, 2, 3, 4 or 5;
Z is CR₈R₉ or absent;
each t is 1, 2, or 3;
each R₁, R₂, R₃, and R₄, independently, is H, amino, hydroxyl, halo, or straight- or branched-chain C₁₋₆ alkyl, C₂₋₆ alkenyl, C₂₋₆ alkynyl, C₁₋₆ heteroalkyl, C₁₋₆ haloalkyl, —CN, —CF₃—OR₁₁, —COR₁₁, —NO₂, —SR₁₁, —NHC(O)R₁, —C(O)NR₁₂R₁₃, —NR₁₂R₃, —NR₁₁C(O)NR₁₂R₁₃, —SO₂NR₁₂R₁₃, —OC(O)R₁₁, —O(CH₂)_{q}NR₁₂R₁₃, or —(CH₂)_{q}NR₁₂R₁₃, where q is an integer from 2 to 6, or R₁ and R₂ together form —NH—N═N— or R₃ and R₄ together form —NH—N═N—;
each R₅, R₆, and R₇, independently, is H, C₁₋₆ alkyl; formyl; C₃₋₆ cycloalkyl; C₅₋₆ aryl, optionally substituted with halo or C₁₋₆ alkyl; or C₅₋₆ heteroaryl, optionally substituted with halo or C₁₋₆ alkyl; each R₈ and R₉, independently, is H or straight- or branched-chain C₁₋₈ alkyl;
R₁₀ is straight- or branched-chain C₁₋₈ alkyl, C₂₋₈ alkenyl, C₂₋₈ alkynyl, C₁₋₈ alkylidene, C₁₋₈ alkoxy, C₁₋₈ heteroalkyl, C₁₋₈ aminoalkyl, C₁₋₈ haloalkyl, C₁₋₈ alkoxycarbonyl, C₁₋₈ hydroxyalkoxy, C₁₋₈ hydroxyalkyl, —SH, C₁₋₈ alkylthio, —O—CH₂—C₅₋₆ aryl, —C(O)—C₅₋₆ aryl substituted with C₁₋₃ alkyl or halo, C₅₋₆ aryl, C₅₋₆ cycloalkyl, C₅₋₆ heteroaryl, C₅₋₆ heterocycloalkyl, —NR₁₂R₁₃, —C(O)NR₁₂R₁₃, —NR₁₁ C(O)NR₁₂R₁₃, —CR₁₁R₁₂R₁₃, —OC(O)R₁₁, —(O)(CH₂)ₛNR₁₂R₁₃ or —(CH₂)_{S}NR₁₂R₁₃, s being an integer from 2 to 8;
R₁₀′ is H, straight- or branched-chain C₁₋₈ alkyl, C₂₋₈ alkenyl, C₂₋₈ alkynyl, C₁₋₈ alkylidene, C₁₋₈ alkoxy, C₁₋₈ heteroalkyl, C₁₋₈ aminoalkyl, C₁₋₈ haloalkyl, C₁₋₈ alkoxycarbonyl, C₁₋₈ hydroxyalkoxy, C₁₋₈ hydroxyalkyl, or C₁₋₈ alkylthio; each R₁₁, independently, is H, straight- or branched-chain C₁₋₈ alkyl, C₂₋₈ alkenyl, C₂₋₈ alkynyl, C₂₋₈ heteroalkyl, C₂₋₈ aminoalkyl, C₂₋₈ haloalkyl, C₁₋₈ alkoxycarbonyl, C₂₋₈ hydroxyalkyl, —C(O)—C₅₋₆ aryl substituted with C₁₋₃ alkyl or halo, C₅₋₆ aryl, C₅₋₆ heteroaryl, C₅₋₆ cycloalkyl, C₅₋₆ heterocycloalkyl, —C(O)NR₁₂R₁₃, —CR₅R₁₂R₁₃, —(CH₂)ₜNR₁₂R₁₃, t is an integer from 2 to 8; and
each R₁₂ and R₁₃, independently, is H, C₁₋₆ alkyl; C₃₋₆ cycloalkyl; C₅₋₆ aryl, optionally substituted with halo or C₁₋₆ alkyl; or C₅₋₆ heteroaryl, optionally substituted with halo or C₁₋₆ alkyl; or R₁₂ and R₁₃ together form a cyclic structure; or a pharmaceutically acceptable salt, ester or prodrug thereof.

In some embodiments, the compounds for use in the present invention are described in U.S. Pat. No. 6,528,529, the disclosure of which is hereby incorporated by reference its entirety, and have the structure of Formula (IV): 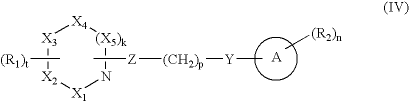
wherein
X₁, X₂, X₃, X₄ and X₅ are selected from C, N and O;
k is 0 or 1;
t is 0, 1 or 2;
R₁ is straight or branched-chain C₁₋₈ alkyl, C₂₋₈ alkenyl, C₂₋₈ alkynyl, C₁₋₈ alkylidene, C₁₋₈ alkoxy, C₁₋₈ heteroalkyl, C₁₋₈ aminoalkyl, C₁₋₈ haloalkyl, C₁₋₈ alkoxycarbonyl, C₁₋₈ hydroxyalkoxy, C₁₋₈ hydroxyalkyl, —SH, C₁₋₈ alkylthio, —O—CH₂—C₅₋₆ aryl, —C(O)—C₅₋₆ aryl substituted with C₁₋₃ alkyl or halo; C₅₋₆ aryl or C₅₋₆ cycloalkyl optionally comprising 1 or more heteroatoms selected from N, S and O; —C(O)NR₃R₄,—NR₃R₄, —NR₃C(O)NR₄R₅, —CR₃R₄, —OC(O)R₃, —(O)(CH₂)ₛNR₃R₄ or —(CH₂)ₛNR₃R₄;
where R₃, R₄ and R₅ are the same or different, each independently being selected from H, C₁₋₆ alkyl; C₅₋₆ aryl optionally comprising 1 or more heteroatoms selected from N, O and S, and optionally substituted with halo or C₁₋₆ alkyl; C₃₋₆ cycloalkyl; or R₃ and R₄ together with the N atom, when present, form a cyclic ring structure comprising 5-6 atoms selected from C, N, S and O; and
s is an integer from 0 to 8;
A is C₅₋₁₂ aryl or C₅₋₇ cycloalkyl, each optionally comprising 1 or more heteroatoms selected from N, S and O;
R₂ is H, amino, hydroxyl, halo, or straight or branched-chain C₁₋₆ alkyl, C₂₋₆ alkenyl, C₂₋₆ alkynyl, C₁₋₆ alkoxy, C₁₋₆ heteroalkyl, C₁₋₆ aminoalkyl, C₁₋₆ haloalkyl, C₁₋₆ alkylthio, C₁₋₆ alkoxycarbonyl, —CN, —CF₃, —OR₃, —COR₃, NO₂, —NHR₃, —NHC(O)R₃, —C(O)NR₃R₄, —NR₃R₄, —NR₃C(O)NR₄R₅, —OC(O)R₃, —C(O)R₃R₄, —O(CH₂)_{q}NR₃, —CNR₃R₄ or —(CH₂)_{q}NR₃R₄;
where q is an integer from 1 to 6;
n is 0, 1, 2, 3 or 4, the groups R₂, when n>1, being the same or different;
p is 0 or an integer from 1 to 5;
Y is O, S, CHOH, —NHC(O)—, —C(O)NH—, —C(O)—, —OC(O)—, NR₇ or —CH═N—, and
R₇ is H or C₁₋₄ alkyl; or absent; and
Z is CR₈R₉ wherein R₈ and R₉ are independently selected from H, and straight or branched chain C₁₋₈ alkyl; or a pharmaceutically acceptable salt, ester or prodrug thereof.

In some embodiments, the compounds for use in the present invention are described in U.S. patent application Ser. No. 10/329,455 (publication number 20030176418), the disclosure of which is hereby incorporated by reference its entirety, and have the structure of Formula (V): 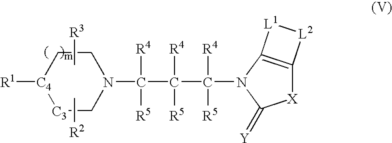
wherein
R¹ is a monoradical selected from the group consisting of optionally substituted C₁₋₆-alkyl, optionally substituted C₂₋₆-alkylidene, optionally substituted C₂₋₆-alkenyl, optionally substituted C₂₋₆-alkynyl, optionally substituted O—C₁₋₆-alkyl, optionally substituted O—C₂₋₆-alkenyl, optionally substituted O—C₂₋₆-akynyl; optionally substituted S—C₁₋₆-alkyl, optionally substituted S—C₂₋₆-alkenyl, optionally substituted S—C₂₋₆-alkynyl;
m is 0, 1 or 2;
C₃-C₄ is CH₂—CH or CH═C or C₄ is CH and C₃ is absent;
R² and R³ are independently selected from the group consisting of hydrogen, optionally substituted C₁₋₆ alkyl, optionally substituted O—C₁₋₆ alkyl, halogen, hydroxy or selected such that R² and R³ together form a ring system;
each R⁴ and R⁵ is independently selected from the group consisting of hydrogen, halogen, hydroxy, optionally substituted C₁₋₆-alkyl, optionally substituted O—C₁₋₆alkyl, optionally substituted aryl-C₁₋₆ alkyl, and optionally substituted arylheteroalkyl;
L¹ and L² are biradicals independently selected from the group consisting of —C(R⁶)═C(R⁷), —C(R⁶)═N—, —N═C(R⁶)—, —S—, —NH— and —O—; wherein only one of L¹ and L² may be selected from the group consisting of —S—, —NH— and —O—;
Y is selected from the group consisting of O, S, and H₂;
X is a biradical selected from the group consisting of —C(R⁶)(R⁷)—C(R⁶)(R⁷)—, —C(R⁶)═C(R⁷)—, —O—C(R⁶)(R⁷)—, C(R⁶)(R⁷)—O—, —S—C(R⁶)(R⁷)—, —C(R⁶)(R⁷)—S—, —N(R^{N})—C(R⁶)(R⁷)—, —C(R⁶)(R⁷)—N(R^{N})—, —C(R⁶)(R⁷)—C(R⁶)(R⁷)—C(R⁶)(R⁷)—, —O—C(R⁶)(R⁷)—C(R⁶)(R⁷)—, S—C(R⁶)(R⁷)—C(R⁶)(R⁷)—, N(R^{N})—C(R⁶)(R⁷)—C(R⁶)(R⁷)—, —C(R⁶)(R⁷)—C(R⁶)(R⁷)—O, —C(R⁶)(R⁷)—C(R⁶)(R⁷)—S, —C(R⁶)(R⁷)—C(R⁶)(R⁷)—N(R^{N})—, —C(R⁶)(R⁷)—C(R⁶)═C(R⁷)—, and —C(R⁶)═C(R⁷)—C(R⁶)(R⁷),
wherein R⁶ and R⁷ are independently selected from the group consisting of hydrogen, halogen, hydroxy, nitro, cyano, NR^{N}R^{N}, N(R^{N})—C(O)N(R^{N}), optionally substituted C₁₋₆-alkyl, C₂₋₆-alkenyl, C₂₋₆-alkynyl, , optionally substituted O—C₁₋₆-alkyl, optionally substituted O-aryl, optionally substituted O—C₂₋₆-alkenyl, optionally substituted O—C₂₋₆-alkynyl
wherein R^{N} is selected from the group consisting of hydrogen, and optionally substituted C₁₋₆-alkyl.

In some embodiments, the compounds for use in the present invention are described in U.S. Provisional Application No. 60/432,692, the disclosure of which is hereby incorporated by reference in its entirety, and have the structure of Formula (VI): 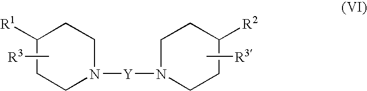
wherein
Y is a biradical of (CR⁴R⁵)ₘ-Z-C(R⁴R⁵)ₙ;
wherein the sum m+n is from 1 to 7;
Z is selected from the group consisting of C(R⁴R⁵), C(O), O, N(R⁶), S, O—C(O), N(R⁶)C(O), C(O)—O, and P; and
R⁴ and R⁵ are independently selected from the group consisting of hydrogen, halogen, hydroxy, nitro, NR⁶N⁶′, optionally substituted aryl, optionally substituted heteroaryl, optionally substituted C₃₋₈-cycloalkyl, optionally substituted heterocyclyl, optionally substituted C₁₋₆-alkyl, optionally substituted C₁₋₆-alkoxy, optionally substituted phenoxy, optionally substituted C₂₋₈-alkenyl and optionally substituted C₂₋₈-alkynyl; and
wherein R¹ and R² are independently selected from the group consisting of optionally substituted aryl, optionally substituted heteroaryl, optionally substituted C₃₋₈-cycloalkyl, optionally substituted heterocyclyl, optionally substituted C₁₋₆-alkyl, optionally substituted C₁₋₆-alkoxy, optionally substituted C₂₋₈-alkenyl and optionally substituted C₂₋₈-alkynyl;
wherein R³ and R³′ are independently selected from the group consisting of hydrogen, halogen, hydroxy, nitro, NR⁶N⁶′, optionally substituted aryl, optionally substituted heteroaryl, optionally substituted C₃₋₈-cycloalkyl, optionally substituted heterocyclyl, optionally substituted C₁₋₆-alkyl, optionally substituted C₁₋₆-alkoxy, optionally substituted C₂₋₈-alkenyl and optionally substituted C₂₋₈-alkynyl; and
R⁶ and R⁶′ are independently selected from the group consisting of hydrogen, optionally substituted aryl, optionally substituted heteroaryl, optionally substituted C₃₋₈-cycloalkyl, optionally substituted heterocyclyl, optionally substituted C₁₋₆-alkyl, optionally substituted C₁₋₆-alkoxy, optionally substituted C₂₋₈-alkenyl and optionally substituted C₂₋₈-alkynyl.

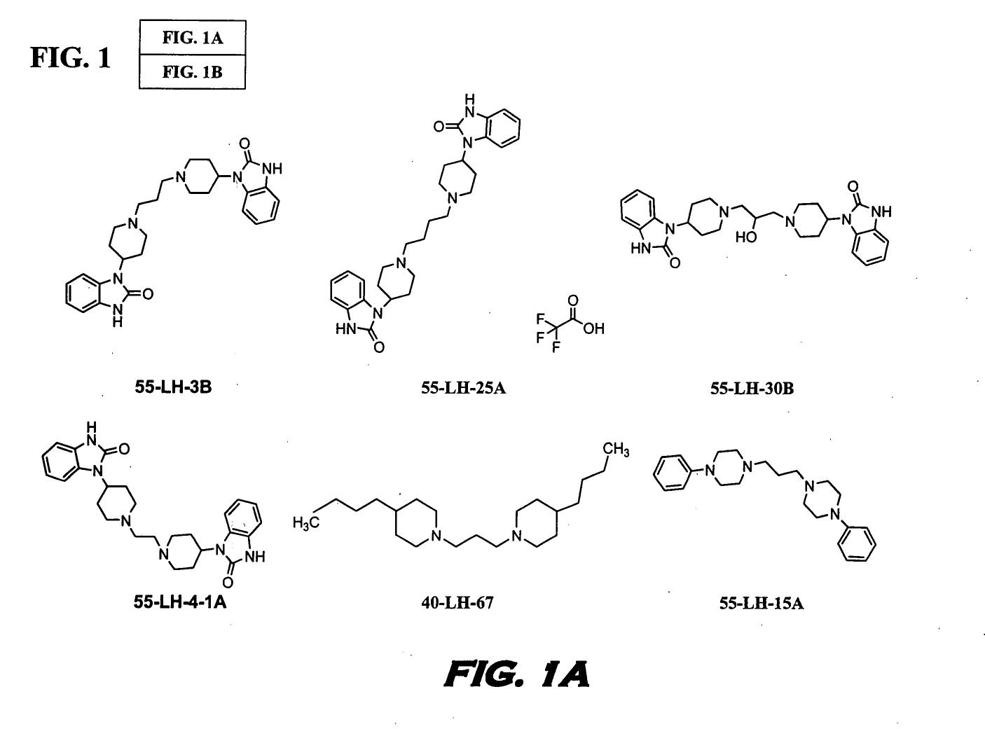

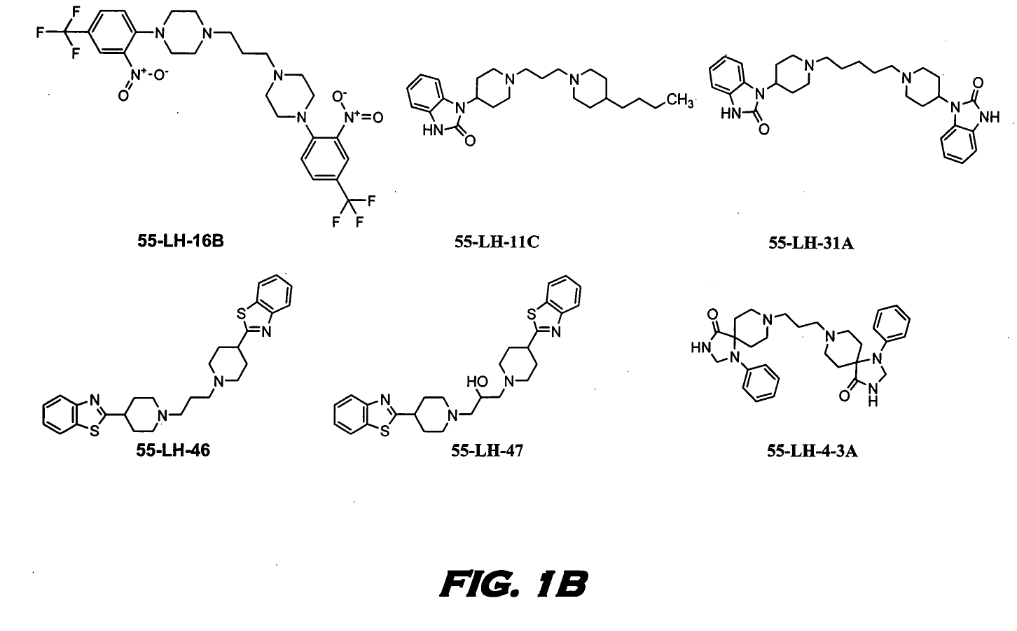

Chemical structures showing specific examples of the compound of Formula (VI) are depicted in FIG. 1. Examples showing the syntheses of these compounds are presented below:

### 1,2-Bis(4-(2-oxobenzimidazolin-1-yl)piperidino)ethane (55-LH-4-1A)

A vial was charged with 4-(2-oxobenzimidazolin-1-yl)piperidine (0.27 g, 1.25 mmol), 1-chloro-2-iodoethane (95 mg, 0.5 mmol), K₂CO₃ (0.17 g, 1.25 mmol) and ethanol (2 mL) and shaken at 60° C. over night. Water and ethyl acetate were added and the product filtered off and dried to give 113 mg of the titled compound.

¹H NMR (DMSO-d₆) δ 1.59-1.66 (m, 4H), 2.06-2.15 (m, 4H), 2.27-2.40 (m, 4H), 2.45 (app s, 4H), 2.99-3.06 (m, 4H), 4.07-4.18 (m, 2H), 6.92-7.00 (app s, 6H), 7.16-7.21 (m, 2H);¹³C NMR (DMSO-d₆) δ 29.4, 50.9, 53.9, 56.3, 109.3, 109.5, 121.1, 121.1, 129.0, 129.9, 154.4. LC-MS[M-H]⁺ 461.4

### 1,4-Bis(4-(2-oxobenzimidazolin-1-yl)piperidino)butane trifluoroacetate (55-LH-25A)

A vial was charged with 4-(2-oxobenzimidazolin-1-yl)piperidine (1.1 g, 5.0 mmol), 4-bromo-1-butanol (0.92 mg, 6.0 mmol), K₂CO₃ (0.86 g, 6.25 mmol) and ethanol (3 mL) and shaken at 60° C. for nine days. Water and ethyl acetate were added and the organic layer was dried (Na₂SO₄), filtered and concentrated. The residue was purified by column chromatography [(SiO₂, 5% NH₄OH in MeOH/EtOAc (1:9)] to give 0.22 mg of 4-(4-(2-oxobenzimidazolin-1-yl)piperidino)butanol (55-LH-10) which was used in the next step without further characterization. LC-MS[M-H]⁺ 290.1

A mixture of 55-LH-10 (0.22 g, 0.78 mmol), DMSO (66 μL, 0.93 mmol) and dichloromethane (1 mL) was cooled to −78° C. and stirred for 0.5 h. Oxalylchloride (73 μL, 0.85 mmol) was added and the mixture was kept at −78° C. for an additional 0.5 h. Triethylamine (0.54 mL, 3.9 mmol) was added and the reaction mixture was allowed to reach room temperature. Water and dichloromethane was added and the organic layer was separated and washed with saturated brine, dried (Na₂SO₄) filered and evaporated. The resulting aldehyde was dissolved in MeOH (2.5 mL) and 4-(2-oxobenzimidazolin-1-yl)piperidine (0.17 g, 0.78 mmol) was added followed by HOAc until pH=4-5. A freshly prepared solution of NaCNBH₃ (54 mg, 0.85 mmol) in MeOH (1 mL) was added and the mixture was stirred at ambient temperature over night. Water and ethyl acetate were added and the organic layer was dried (Na₂SO₄), filtered and concentrated. The residue was dissolved in aqueous HCI (1N) and purified by preparative HPLC [Luna column (21.2×250 mm, 15 μm C18(2), 0.1% TFA in H₂O/0.1% TFA in CH₃CN/H₂O (8:2) (9:1 gradient to 0:100)]. The pure compound precipitated from water as the trifluoroacetate salt (24 mg). ¹H NMR (CD₃OD) δ 1.89-1.96 (m, 4H), 2.06-2.14 (m, 4H), 2.79-2.93 (m, 4H), 3.09-3.32 (m, 8H), 3.73-3.3.82 (m, 4H), 4.55-4.65 (m, 2H), 7.05-7.15 (m, 6H), 7.28-7.33 (m, 2H); LC-MS[M-H]⁺ 4.89.2

### 5-(4-(2-Oxobenzimidazolin-1-yl)piperidino)pentanol (55-LH-27A)

Compound 55-LH-27 was prepared according to the procedure used for the preparation of 55-LH-10 using 5-bromo-1-pentanol (1.0 g, 6.0 mmol). After 10 days at 60° C., water was added and the product was filtered off to yield 0.79 g of the titled compound.

¹H NMR (CD₃OD) δ 1.35-1.50 (m, 2H), 1.55-1-65 (m, 4H), 1.70-1.85 (m, 2H), 2.10-2.25 (m, 2H), 2.40-2.60 (m, 4H), 3.05-3.15 (m, 2H), 3.50-3.60 (m, 2H), 4.25-4.40 (m, 1H), 7.05-7.15 (m, 3H), 7.35-7.45 (m, 1H); ¹³C NMR (CD₃OD) δ 22.8, 26.5, 28.4, 32.3, 50.7, 53.1, 58.4, 61.6, 109.4, 109.6, 121.0, 121.3, 128.5, 129.1, 155.1; LC-MS[M-H]⁺ 304.3

### 1,5-Bis(4-(2-oxobenzimidazolin-1-yl)piperidino)pentane (55-LH-31A)

Compound (55-LH-31A) was prepared according to the procedure used for the preparation of 55-LH-25A using 55-LH-27A (0.30 g, 1.0 mmol). The residue was purified by preparative HPLC [Luna column (21.2×250 mm, 15 μm C18(2), 0.1% TFA in H₂O/0.1% TFA in CH₃CN/H₂O (8:2) 9:1 gradient to 0:100)]. The solvent was evaporated and the residue was dissolved in water and dichloromethane. Ammonium hydroxide was added until pH=10 and the organic layer was dried (Na₂SO₄), filtered and concentrated. The residue was dissolved in MeOH and trifluoroacetic acid (5 μL) was added. The trifluoroacetate salt was purified on preparative HPLC [Luna column (21.2×250 mm, 15 μm C18(2), 0.1% TFA in H₂O/0.1% TFA in CH₃CN/H₂O (8:2) (9:1 gradient to 0:100)]. The solvent was evaporated and NH₄OH was added to the aqueous solution until pH=10. The product was filtered off and dried to give 47 mg of the titled compound.

¹H NMR (CD₃OD) δ 1.37-1.46 (m, 2H), 1.59-1-68 (m, 4H), 1.74-1.82 (m, 4H), 2.16-2.25 (m, 4H), 2.44-2.60 (m, 8H), 3.12-3.20 (m, 4H), 4.28-4.38 (m, 2H), 7.02-7.08 (m, 6H), 7.36-7.41 (m, 2H); ¹³C NMR (CD₃OD) δ 25.6, 26.6, 28.4, 50.7, 53.1, 58.3, 109.4, 109.6, 121.0, 121.3, 128.5, 129.1, 155.1; LC-MS[M-H]⁺ 503.1

### 1,3-Bis(4-(2-oxobenzimidazolin-1-yl)piperidino)propane (55-LH-3B)

A vial was charged with 4-(2-oxobenzimidazolin-1-yl)piperidine (1.09 g, 5 mmol), 1-chloro-3-iodopropane (250 μL, 2 mmol), K₂CO₃ (0.69 g, 5 mmol) and ethanol (10 mL) and shaken at 60° C. for six days. Water, ethyl acetate and MeOH were added. The organic layer was evaporated and the residue was purified by column chromatography [(SiO₂, 5% NH₄OH in MeOH/ethyl acetate (1:9)] and then by preparative HPLC [Luna column (21.2×250 mm, 15 μm C18(2), 0.1% TFA in H₂O/0.1% TFA in CH₃CN/H₂O (8:2) (9:1 gradient to 0:100)]. The solvent was evaporated and NH₄OH was added to the aqueous solution until pH=10. The product was filtered off, washed with water and dried to give 235 mg of the titled compound.

¹H NMR (CD₃OD) δ 1.76-1.88 (m, 6H), 2.20-2.28 (m, 4H), 2.48-2.62 (m, 8H), 3.14-3.22 (m, 4H), 4.28-4.38 (m, 2H), 7.02-7.09 (m, 6H), 7.35-7.40 (m, 2H); ¹³C NMR (CD₃OD) δ 6 24.0, 28.4, 50.7, 53.1, 56.3, 109.4, 109.5, 121.1, 121.3, 128.5, 128.2, 155.1; LC-MS[M-H]⁺ 475.4

### 1,3-Bis(1-phenyl-4-oxo-1,3,8-triazaspiro[4,5]decan-8-yl)propane (55-LH-4-3A)

A vial was charged with 1-phenyl-1,3,8-triazaspiro[4,5]decan-4-one (0.29 g, 1.25 mmol), 1-chloro-3-iodopropane (0.10 g, 0.5 mmol), K₂CO₃ (0.17 g, 1.25 mmol) and ethanol (2 mL) and shaken at 60° C. over night. Water and ethyl acetate were added. The product was filtered off and dried to give 154 mg of the titled compound.

¹H NMR (CD₃OD) δ 1.69-1.83 (m, 6H), 2.43-2.49 (m, 4H), 2.57.2.67 (m, 4H), 2.84-2.90 (m, 8H), 4.68 (s, 4H), 6.82-6.87 (m, 2H), 6.99-7.04 (m, 4H), 7.22.7.27 (m, 4H; ¹³C NMR (CD₃OD) δ 23.9, 28.8, 49.5, 56.5, 59.4, 59.7, 116.5, 119.4, 128.9, 143.6, 178.2; LC-MS[M-H]⁺ 503.4

### 3-[4-(2-Oxobenzimidazolin-1-y)piperidino]-1-(4-butylpiperidino)propane (55-LH-11C)

A vial was charged with 4-(2-oxobenzimidazolin-1-yl)piperidine (0.13 g, 0.6 mmol), 1-chloro-3-iodopropane (64 μL, 0.6 mmol), K₂CO₃ (0.173 g, 1.25 mmol) and ethanol (2 mL) and shaken at 60° C. for five days. 4-Butylpiperidine (0.85 g, 0.6 mmol) was added and the mixture was shaken at 60° C. for two additional days. Water and ethyl acetate were added. The organic layer was dried (Na₂SO₄), filtered and concentrated. The residue was purified by column chromatography [(SiO)₂, 5% NH₄OH in MeOH/ethyl acetate (1:9)], preparative LC-MS [Waters symmetry C18 (19×50 mm, 5 μm particles), 0.15% TFA in H₂O/0.15% TFA in CH₃CN/H₂O (95:5) (9:1 gradient to 0:100)] and preparative HPLC [Luna column (21.2×250 mm, 15 μm C18(2), 0.1% TFA in H₂O/0.1% TFA in CH₃CN/H₂O (8:2) (9:1 gradient to 0:100)]. The solvent was evaporated and NH₄OH was added to the aqueous solution to pH=10. The organic layer was dried (Na₂SO₄) filtered and evaporated to yield 11.4 mg of the titled compound.

¹H NMR (CD₃OD) δ 0.88-0.93 (m, 3H), 1.18-1.34 (m, 9H), 1.68-1.83, (m, 6H), 1.97-2.06 (m, 2H), 2.15-2.24 (m, 2H) 2.38-2.58 (m, 6H), 2.94-3.01 (m, 2H), 3.10-3.17 (m, 2H), 4.26-4.36 (m, 1H), 7.02-7.08 (m, 3H), 7.36-7.39 (m, 1H); ¹³C NMR (CD₃OD) δ 13.2, 22.8, 23.7, 28.4, 28.9, 29.7, 35.6, 36.2, 50.8, 53.1, 53.9, 56.4, 56.9, 109.4, 109.5, 121.0, 121.3, 128.5, 129.2, 155.1; LC-MS[M-H]⁺ 399.3

### 1,3-Bis (4-butylpiperidino)propane (40-LH-67)

A vial was charged with 4-butylpiperidine (0.13 g, 0.9 mmol), 1-chloro-3-iodopropane (107 μL, 1.0 mmol), K₂CO₃(0.35 g, 2.5 mmol) and ethanol (4 mL) and shaken at 60° C. over night. Water and ethyl acetate were added. The organic layer was evaporated and the residue was purified by preparative LC-MS [Waters symmetry C18 (19×50 mm, 5 μparticles), 0.15% TFA in H₂O/0.15% TFA in CH₃CN/H₂O (95:5) (9:1 gradient to 0:100)] to give 6.4 mg of the titled compound.

¹H NMR (CDCI₃) δ 0.84-1.10 (m, 6H), 1.16-1.32 (m, 18H), 1.62-1.74 (m, 6H), 1.82-1.91 (m, 4H), 2.26-2.32 (m, 4H), 2.86-2.92 (m, 4H); ¹³C NMR (CDCl₃) δ 14.3, 23.1, 25.0, 29.3, 32.7, 36.1, 36.6, 54.4, 57.6; LC-MS[M-H]⁺ 323.4

### 1,3-Bis[4-(2-oxobenzimidazolin-1-yl)piperidino]-2-propanol (55-LH-30B)

A vial was charged with 4-(2-oxobenzimidazolin-1-yl)piperidine (0.44 g, 2 mmol), epichlorohydrin (78 μL, 1 mmol), K₂CO₃ (0.35 g, 2.5 mmol) and ethanol (3 mL) and shaken at 60° C. for 19 days. Water was added and the product was filtered off to give 400 mg crude product of which 150 mg was purified by preparative HPLC [Luna column (21.2×250 mm, 15 μm C18(2), 0.1% TFA in H₂O/0.1% TFA in CH₃CN/H₂O (8:2) (9:1 gradient to 0:100)] to give 50 mg of the titled compound.

¹H NMR (CD₃OD) δ 1.76-1.84 (m, 4H), 2.32-2.66 (m, 12H), 3.20-3.28 (m, 4H), 4.01-4.08 (m, 1H), 4.28-4.38 (m, 2H), 7.02-7.09 (m, 6H), 7.35-7.40 (m, 2H); ¹³C NMR (CD₃OD) δ 28.4, 28.4, 50.7, 53.2, 54.2, 62.6, 65.4 109.4, 109.5, 121.1, 121.3, 128.5, 128.2, 155.1; LC-MS[M-H]⁺ 491.0

### 1,3-Bis(4-phenyl-1-piperazinyl)propane (55-LH-15)

A vial was charged with 4-phenylpiperazine (191 μL, 1.25 mmol), 1-chloro-3-iodopropane (54 μL, 0.5 mmol), K₂CO₃ (0.17 g, 1.25 mmol) and ethanol (3 mL) and shaken at 60° C. for five days. Water was added and the product was filtered off and dried to give 145 mg of the titled compound.

¹H NMR (CD₃OD) δ 1.76-1.86 (m, 2H), 2.44-2.51 (m, 4H), 2.63-2.69 (m, 8H), 3.17-3.22 (m,8H), 6.81-686 (m, 2H), 6.94-6.99 (m, 4H), 7.20-7.26 (m, 4H); ¹³C NMR (CD₃OD) δ 23.4, 49.1, 53.1, 56.5, 116.3, 120.0, 128.9, 151.5; LC-MS[M-H]⁺ 365.2

### 1,3-Bis(4-(2-nitro-4-trifluoromethylphenyl)-1-piperazinyl)propane (55-LH-16B)

A vial was charged with (4-(2-nitro-4-trifluoromethylphenyl)piperazine (0.34 g, 1.25 mmol), 1-chloro-3-iodopropane (54 μL, 0.5 mmol), K₂CO₃ (0.17 g, 1.25 mmol) and ethanol (3 mL) and shaken at 60° C. for five days. Water was added and the product was filtered off and dried. Recrystallization (2-propanol) gave 226 mg of the titled compound.

¹H NMR (CD₃OD) δ 1.74-1.83 (m, 2H), 2.46-2.52 (m, 4H), 2.61-2.66 (m, 8H), 3.18-3.23 (m, 8H), 7.37-7.42 (m, 2H), 7.76-7.79 (m, 2H), 8.04-8.07 (m, 2H); ¹³C NMR (CD₃OD) δ 23.4, 50.4, 52.7, 56.2, 121.3, 121.9, 123.5, 123.8, 129.9, 141.2, 148.0; LC-MS[M-H]⁺ 591.2

### 1,3-Bis(4-(2-benzothiazolyl)piperidino)propane (55-LH-46)

A vial was charged with (4-(2-benzothiazolyl)piperdine (0.15 g, 0.69 mmol), 1-chloro-3-iodopropane (36 μL, 0.34 mmol), K₂CO₃ (97 mg, 0.70 mmol) and ethanol (2 mL) and shaken at 60° C. for five days. Water was added and the product was filtered off and dried to give 138 mg of the titled compound.

¹H NMR (CD₃OD) δ 1.74-1.84 (m, 2H), 1.90-2.03 (m, 4H), 2.14-2.26 (m, 8H), 2.41-2.48 (m, 4H), 3.04-3.20 (m, 6H), 7.36-7.42 (m, 2H), 7.44-7.51 (m, 2H), 7.89-7.96 (m, 4H); ¹³C NMR (CD₃OD) δ 23.632.0, 41.2, 53.2, 56.6, 121.7, 122.0, 125.0, 126.1, 134.4, 152.8, 176.8; LC-MS[M-H]⁺ 477.1

### 1,3-Bis(4-(2-benzothiazolyl)piperidino)-2-propanol (55-LH-47)

A vial was charged with (4-(2-benzothiazolyl)piperdine (0.15 g, 0.69 mmol), epichlorohydrin (27 μL, 0.34 mmol), K₂CO₃ (97 mg, 0.70 mmol) and ethanol (2 mL) and shaken at 60° C. for five days. Water was added and the product was filtered off and dried to give 140 mg of the titled compound.

¹H NMR (CD₃OD) δ 1.90-2.05 (m, 4H), 2.10-2.20 (m, 4H), 2.21-2.52 (m, 8H), 3.07-3.18 (m, 6H), 3.96-4.04 (m, 1H), 7.35-7.42 (m, 2H), 7.44-7.51 (m, 2H), 7.88-7.96 (m, 4H); ¹³C NMR (CD₃OD) δ 32.2, 32.2, 41.2, 53.4, 54.2, 63.2, 65.7, 121.7, 122.0, 125.0, 126.1, 134.4, 152.8, 177.1; LC-MS[M-H]⁺ 493.1

In some embodiments, the compounds for use in the present invention include the compound of Formula VII, which is disclosed in U.S. Pat. No. 6,627,645, 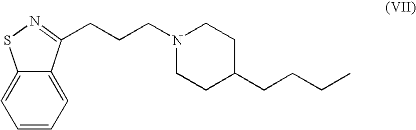
the disclosure of which is hereby incorporated by reference in its entirety, and the compounds of Formulas VIII and IX, which are disclosed in U.S. application Ser. No. 10/329,455 (publication number 20030176418), the disclosure of which is hereby incorporated by reference in its entirety.

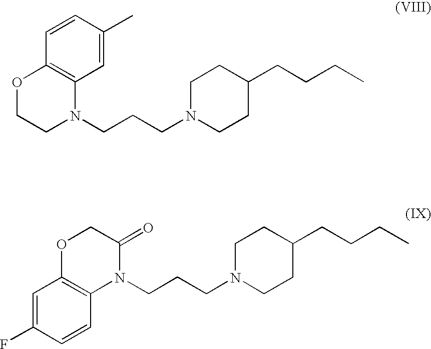

Certain of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts such as hydrochloride, hydrobromide, phosphate, sulphate, acetate, citrate, lactate, tartrate, maleate, fumarate, mandelate and oxalate; and inorganic and organic base addition salts with bases such as sodium hydroxy and Tris(hydroxymethyl)aminomethane (TRIS, tromethane).

In addition to administering a compound as a raw chemical, the compounds of the invention may be administered as part of a pharmaceutical preparation containing suitable pharmaceutically acceptable carriers comprising excipients and auxiliaries which facilitate processing of the compounds into preparations which can be used pharmaceutically. Preferably, the preparations, particularly those preparations which can be administered orally or topically and which can be used for the preferred type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by injection, topically or orally, contain from about 0.01 to 99 percent, preferably from about 0.25 to 75 percent of active compound(s), together with the excipient.

Also included within the scope of the present invention are the non-toxic pharmaceutically acceptable salts of the compounds of the present invention. Acid addition salts are formed by mixing a solution of the M1 receptor agonists described herein with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, and the like. Basic salts are formed by mixing a solution of the particular M1 receptor described herein with a solution of a pharmaceutically acceptable non-toxic base such as sodium hydroxide, potassium hydroxide, choline hydroxide, sodium carbonate Tris and the like.

The pharmaceutical compositions of the invention may be administered to any animal which may experience the beneficial effects of the compounds of the invention. Foremost among such animals are mammals, for example, humans, although the invention is not intended to be so limited.

The M1receptor agonists and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the MI receptor agonists described herein are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropymethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, enemas or suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances which increase the viscosity of the suspension include, for example, sodium carboxymethyl cellulose, sorbitol, and/or dextran. Optionally, the suspension may also contain stabilizers.

Compositions within the scope of this invention include all compositions wherein the compounds described herein are contained in an amount effective to achieve its intended purpose. While individual needs vary, determination of optimal ranges of effective amounts of each component is within the skill of the art. Typically, the compounds may be administered to mammals, for example, humans, orally at a dose of 0.0025 to 50 mg/kg, or an equivalent amount of the pharmaceutically acceptable salt thereof, per day of the body weight of the mammal being treated. Preferably, about 0.01 to about 10 mg/kg is orally administered. For intramuscular injection, the dose is generally about one-half of the oral dose.

The unit oral dose may comprise from about 0.01 to about 50 mg, preferably about 0.1 to about 10 mg of the compound. The unit dose may be administered one or more times daily as one or more tablets each containing from about 0.1 to about 10, conveniently about 0.25 to 50 mg of the compound or its solvates.

In a topical formulation, the compound may be present at a concentration of about 0.01 to 100 mg per gram of carrier. In a preferred embodiment, the compound is present at a concentration of about 0.07-1.0 mg/ml, more preferably, about 0.1-0.5 mg/ml, most preferably, about 0.4 mg/ml.

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### EXAMPLE 1

The functional receptor assay, Receptor Selection and Amplification Technology (R-SAT), essentially as disclosed in U.S. Pat. Nos. 5,707,798, 5,912,132, and 5,955,281, which are all hereby incorporated by reference in their entirety, was used to investigate the pharmacological properties of known and novel muscarinic agonists. Accordingly, xanomeline, oxotremorine, milameline, and the compounds of formulas VII, VIII, and IX were tested.

These experiments have provided a molecular profile, or fingerprint, for each of these agents across the most meaningful receptors, the M(1) and M(2) muscarinic receptor subtypes. As can be seen in Table 1, the three reference agents, xanomeline, oxotremorine and milameline, are potent and efficacious full agonists at both the M(1) and M(2) receptor subtypes. In contrast, the compounds of Formulas VII, VIII, and IX are potent and efficacious M(1) agonist but only weak partial agonists at M(2) receptors.

TABLE 1
Comparison of Reference Muscarinic Agonists with ACADIA's M(1) Agonists
in R-SAT Assays and Rodent Models of Pain

| | M1 | | M2 | | Acute | | |
|---|---|---|---|---|---|---|---|
| Compounds | pEC50 | % efficacy | pEC50 | % efficacy | pain | Antihyperalgesic | Antiallodynic |
| Xanomeline | 7.2 | 121.0 | 6.5 | 109.0 | 10.0 | 10.0 | 10.0 |
| Oxotremorine | 7.2 | 91.0 | 7.8 | 104.0 | 0.3 | 0.3 | 0.3 |
| Milameline | 6.4 | 90.0 | 6.2 | 110.0 | 1.0 | 0.3 | 0.3 |
| Formula (VII) | 7.1 | 85.0 | 5.9 | 36.0 | NA | 10.0 | 10.0 |
| Formula | 7.7 | 81.0 | 6.3 | 39.0 | NA | 10.0 | 30.0 |
| (VIII) | | | | | | | |
| Formula (IX) | 7.5 | 79.0 | 6.3 | 48.0 | NA | 10.0 | 17.8 |

% efficacy is relative to carbachol
NA = not active at the highest tested dose of 30 mg/kg
All in vivo results are expressed as the minimal effective dose in mg/kg

CCI/Thermal Hyperalgesia

Rats were anesthetized under aseptic and heated conditions using a combination of 1.6 ml ketamine (100 mg/ml) and 1.6 ml xylazine (100 mg/ml) in 6.8 ml 0.9% saline at a volume of 0.1 ml/100 g. The left quadriceps was shaved and scrubbed thoroughly with an iodine solution. The sciatic nerve was exposed at the level of the sciatic notch distally to the sciatic trifurcation. The nerve was very carefully freed from the underlying muscle and connective tissue without causing trauma to the nerve itself. Using 4-0 chromic catgut suture material, four semi-loose ligatures were tied around the sciatic nerve starting at the most proximal level, next to the sciatic notch, spaced roughly 1 mm apart and ending proximal to the sciatic trifurcation. Under magnification the ligatures were tightened until a slight twitch was observed in the animals left paw or musculature surrounding the nerve. The muscular incision was closed with 4-0 silk suture material and the skin was stapled with wound clips. The animals were closely observed until they recovered completely from the anesthetic. The surgery was the same for the hyperalgesia and allodynia experiments.

For hyperalgesia testing, rats were placed in a tinted plastic box on top of a clear glass, temperature-regulated floor maintained at 31±1° C. The floor contained a focal radiant heat source (halogen projection lamp CXL/CXP, 50 W, 8 v, USHIO, Tokyo). The heat source was moveable beneath the glass and had a radiant beam of approximately 3 mm in diameter, that could be positioned under the plantar surface of the rat hind paw.

To initiate the test, rats were placed in the tinted boxes and allowed 10-20 minutes to acclimate to the new environment. The radiant heat source was then positioned under the plantar surface of the hind paw. Upon activation of the heat source, a timer was simultaneously triggered. Upon reflex movement of the hind paw, a motion sensor was activated stopping the timer and inactivating the heat source. The thermal source was adjusted so that the average response latency for an uninjured animal was no greater than 20 seconds. Each rat had two days of pre-operative baseline latency measurements in which the left rear hind paw plantar surface was measured three to four times. Two to three left postoperative baseline latency measurements were taken before and after the treatment was given. Postoperative day 2 and 4 measurements yielded the greatest degree of hyperalgesia and thus were utilized in this assay. Each animal was tested twice with at least 48 hours separating each test.

Thermal hyperalgesia developed in the surgical-treated left paw as evidenced by a decrease in paw withdrawal latencies to a thermal stimulus. The maximal hyperalgesia occurred on post-operative days 2 through 4. Paw withdrawal latencies on the surgically-treated left side gradually returned to baseline levels over the course of 5 to 12 days post-surgery. The surgically untreated right paw was not significantly affected by surgery as evidenced by similar paw withdrawal latencies throughout the 12 days of testing.

Vehicle administration in each group did not alter the thermal hyperalgesia. In contrast, the reference muscarinic agonists dose dependently reversed thermal hyperalgesia (Table 1). Xanomeline reversed the thermal hyperalgesia [F (2,15)=57.43, p<0.001]. Dunnett's post-hoc comparison revealed that xanomeline reversed thermal hyperalgesia at 10 mg/kg (p<0.001), but not 3 mg/kg (p>0.05) relative to vehicle. Oxotremorine also reversed thermal hyperalgesia [F (2,11)=13.74, p=0.0018]. Post-hoc comparison demonstrated that paw withdrawal latencies after oxotremorine administration at 1 mg/kg (18.468±1.532 s; p<0.001) and 0.3 mg/kg (13.683 s±1.36; p<0.05) were statistically different from vehicle. Significant anti-hyperalgesia also was observed with milameline, [F (2,14)=106.9, p<0.0001], with doses of 1 mg/kg p (p<0.001) and 0.3 mg/kg (p<0.0001) significantly increasing paw withdrawal latencies. In comparison, morphine [F (3,20)=15.55, p<0.0001] caused significant anti-hyperalgesia at doses of 1 mg/kg (16.856 s±1.05, p<0.01) and 3 mg/kg (16.817 s±1.6, p<0.01).

Like the reference muscarinic agonists, compounds of Formulas VII, VIII, and IX dose dependently reversed thermal hyperalgesia: Formula VII, F(4,29)=13.2, p<0.0001; Formula VIII, F(2,23)=6.066, p=0.0041; Formula IX, [F (4,24)=14.51, p<0.0001]. Dunnett's post-hoc comparison revealed that the compounds of Formulas VII, VIII, and IX reversed thermal hyperalgesia at 10 mg/kg (p<0.001).

CCI/Tactile Allodynia

The onset and duration of significant mechanical allodynia post CCI surgery is approximately 10-14 days and lasts for roughly two months. Within this allodynic time frame, and for each specific allodynia experiment, pre and post drug administration measurements were taken with seven von Frey hairs which are designated by log (10* force required to bend hair, mg) and ranged from 2-26 grams (#'s 4.31-5.46). Each hair was pressed perpendicularly against the left injured plantar mid-hind paw surface with sufficient force to cause a slight bending, and was held for 6-8 seconds starting with the thinnest gauged hair and working up to the thickest. A positive response was recorded when the injured paw was sharply withdrawn, and this response was confirmed as positive by testing the next thickest gauged hair for the same response. Only when a response was seen twice was the score accepted. If the maximum gram force of 26 was reached without a response, this was considered the peak threshold cutoff for allodynic behavior and the score was recorded. Animals were considered allodynic when the post surgery baseline measurements were 6 grams and below. Two baseline days of measurements were taken with one round of testing occurring per day. On the day of drug testing, one round of baseline measurements were taken, the appropriate pretreatment was administered i.p. and a second round of measurements were recorded. Each animal was utilized in multiple experiments, with one treatment per experiment, and an appropriate washout period in between experiments.

Significant tactile allodynia was seen starting on day 8 and continuing through day 35-post surgery. Assessment of tactile responsivity after these muscarinic agonists was performed within these post surgical time points. In the vehicle treated group post injury pre-treatment scores were not statistically significant from base line, [F (2,95)=1.275, p>0.05]. The three reference muscarinic agonist also dose dependently reversed tactile allodynia. Xanomeline reversed tactile allodynia, [F (3,22)=12.58, p<0.0001] at doses of 10.0 and 30 mg/kg (p<0.01). Oxotremorine also reversed tactile allodynia [F (3,19)=32.49, p<0.0001] at a dose 0.3 mg/kg (p<0.05) and 1 mg/kg (p<0.01). The results for CI-979 were similar to what was seen with the other muscarinic agonists, [F (2,14)=24.38, p<0.0001]. At a doses of 0.3 mg/kg (p<0.05) and 1 mg/kg (p<0.01), CI-979 increased tactile thresholds. Morphine elicited anti-allodynia in a manner similar to these muscarinic agonists, [F (2,17)=6.257, p=0.0106].

Again, like the reference muscarinic agonists, the compounds of Formulas VII, VIII, and IX dose dependently reversed tactile allodynia: Formula VII, F(3,20)=29.11, p<0.0001; Formula VIII, F(3,23)=11.764, p<0.0001; Formula IX, F(4,28)=7.569, p=0.0004. Dunnett's post-hoc comparison revealed that Formula VII reversed tactile allodynia at 10 mg/kg (p<0.001), Formula VIII reversed tactile allodynia at 30 mg/kg (p=0.08) and Formula IX reversed tactile allodynia at 17.8 mg/kg (p<0.001).

Acute Thermal Analgesia

Water was heated and maintained at 55° C.±1° C. with a probe regulated hot plate. Female rats weighing approximately 200 g -250 g were acclimated days in advance by placing them into and removing them from a plastic rat restrainer. On the day of the experiment each rat was placed in the restrainer 1 minute before the test was performed. Roughly one inch of the tail was submerged into the water as a timer was initiated. Once the tail was completely removed from the water, the timer was stopped and the time was recorded. If the animal did not respond within 10 seconds, the experimenter removed the tail from the heated water and recorded this as the maximum score. One round of baseline measurements were collected. The test compound was administered and after the appropriate pretreatment interval, the procedure was repeated. Each animal was utilized in multiple experiments, with one treatment per experiment, and an appropriate washout period of at least 48 hours between experiments. The effects of test compounds on acute nociception are shown in Table 1. The pre-treatment baseline tail withdrawal latency average was 2.281 s±0.25. Vehicle administration did not alter tail withdrawal latencies with an average latency of 3.16 s±0.21. Xanomeline [F (2,16)=4.952, p<0.05], oxotremorine [F (2,17)=20.50, p<0.05], and milameline [F (2,17)=19.25, p<0.05produced significant antinociception. Xanomeline only was active at the 10.0 mg/kg dose, oxotremorine at the 0.3 mg/kg and 1.0 mg/kg doses and milameline at the 1.0 mg/kg dose. At a dose of 10 mg/kg, morphine [F(3,23)=5.903, p<0.01] was antinociceptive.

Surprisingly, the compounds of Formulas VII, VIII, and IX were found to be not active in alleviating acute thermal pain (Table 1). Thus, the compounds of Formulas VII, VII, and IX reverse chronic neuropathic pain but are not acutely antinociceptive.

### EXAMPLE 2

Muscarinic Side Effects

All of the reference muscarinic receptor agonists tested produced cholinergic side effects as shown in Table 2. The number of animals exhibiting each side effect at each dose is shown compared to the number of animals tested (N). Xanomeline at a dose of 30 mg/kg produced diarrhea, salivation, and lethargy in all animals tested at this dose, whereas the lower dose of 10 mg/kg only produced diarrhea in 2 of 11 animals tested. Oxotremorine at a dose of 1 mg/kg produced all five of the measured muscarinic side effects in the majority of the rats, where as 0.3 mg/kg produced only diarrhea, salivation and lethargy. Milameline at 1 mg/kg, like oxotremorine, produced four of the measured side effects but not tremors, where as the lower dose of 0.3 mg/kg produced predominately diarrhea. In contrast, none of the compounds of Formulas VII, VII, or IX produced any of these side-effects at doses between 3.0 mg/kg and 30 mg/kg. Thus, the reference muscarinic agonists produce severe muscarinic mediated side-effects at doses similar to those required to produce efficacy in these pain models whereas the compounds of Formulas VII, VIII, and IX do not produce these side-effects at doses that efficacious in the neuropathic pain models.

TABLE 2
Side effect profile of reference muscarinic agonists

| | | Diar- | Sali- | Trem- | | Leth- |
|---|---|---|---|---|---|---|
| Compounds | N | rhea | vation | or | Chromodaccyhrea | argy |
| Xanomeline | | | | | | |
| 3 mg/kg | 6 | 0 | 0 | 0 | 0 | 0 |
| 10 mg/kg | 11 | 2 | 0 | 0 | 0 | 0 |
| 30 mg/kg | 6 | 6 | 6 | 0 | 0 | 6 |
| Oxotremorine | | | | | | |
| 0.1 mg/kg | 12 | 1 | 0 | 0 | 0 | 0 |
| 0.3 mg/kg | 15 | 7 | 9 | 0 | 2 | 0 |
| 1 mg/kg | 21 | 18 | 16 | 6 | 8 | 18 |
| Milameline | | | | | | |
| 0.1 mg/kg | 6 | 0 | 0 | 0 | 0 | 0 |
| 0.3 mg/kg | 16 | 9 | 1 | 0 | 0 | 0 |
| 1 mg/kg | 16 | 15 | 9 | 0 | 13 | 16 |
| Vehicle | 32 | 0 | 0 | 0 | 0 | 0 |

### EXAMPLE 3

Partial Sciatic Ligation (PSL) Surgery/Tactile Allodynia

Male mice (C57B1/6) were anesthetized using 1% Isoflurane (1 Lpm) inhalation anesthetic under aseptic and heated conditions. The left quadriceps was shaved and scrubbed thoroughly with an iodine solution. The sciatic notch was palpated and an incision made from the notch to mid quadriceps. The sciatic nerve was exposed at the level of the sciatic notch distally to the sciatic trifurcation. The nerve was carefully freed from the underlying muscle and connective tissue without causing trauma to the nerve itself. When necessary sterile saline was applied to the exposed tissue to prevent it from drying out. Using 10-0 polypropelene blue monofilament suture, the sciatic nerve was perforated immediately distal to the sciatic notch and ligation tied to occlude ⅓ to ½ of the sciatic nerve. Under magnification the ligature was tightened until a slight twitch was observed in the animals left paw. The muscular incision was closed, when necessary, with 7-0 polypropelene suture and the skin was stapled with wound clips. Post-opertative buprenex was administered at 0.075 mg/kg SC. The animals were closely observed until they recovered completely from the anesthetic.

The onset for significant tactile allodynia post PSL surgery is approximately 4-6 days and lasts for roughly one month. Within this allodynic time frame, and for each specific allodynia experiment, pre and post drug administration measurements were taken with eight von Frey hairs which are designated by log (10* force required to bend hair) and ranged from 0.07.-4 grams. Each hair was pressed perpendicularly against the left injured plantar mid hind paw surface with sufficient force to cause a slight bend in the hair, and was held for 6-8 seconds starting with the thinnest gauged hair and working up to the thickest. A positive response was recorded when the injured paw was sharply withdrawn, and this response was confirmed positive by testing the next thickest gauged hair for the same response. Only when this response was seen twice was the score accepted from the hair that produced the initial behavioral response. If the maximum gram force of 10 was reached without a response, this was considered the peak threshold cutoff for allodynic behavior and the score was recorded. Animals were considered allodynic when the post surgery baseline measurements were ˜60% of presurgical baseline measurements. Two baseline days of measurements were taken with one round of testing occurring per day. On the day of drug testing, one round of baseline measurements were taken, the appropriate pretreatment was administered i.p. or sc., and a second round of measurements were recorded. Each animal was utilized in multiple experiments, with one treatment per experiment, and an appropriate washout period in between experiments.

Muscarinic M(1) receptor knockout (KO) mice did not differ from wild type (WT) with respect to pre-surgery tactile sensitivity (t=1.094, df=15, p=0.2913) nor with respect to post-surgery allodynia (t=0.2338, df=15, p=0.8183). Both M(1) KO (t=5.765, df=7, p=0.0007) and WT (t=3.551, df=8, p=0.0075) mice developed robust tactile allodynia following PSL surgery. However, the compound of Formula IX at 30 mg/kg significantly alleviated the tactile allodynia in WT mice, but the effects of the compound of Formula IX was completely abolished in M(1) KO mice, confirming the role for M(1) receptors in neuropathic pain in vivo. Control tactile sensitivity before surgery (Pre-PSL) and after surgery (PSL) are shown in FIG. 1 for comparison to sensitivity after treatment with the compound of Formula IX in wild type (+/+) and M(1) receptor knockout (−/−) mice.

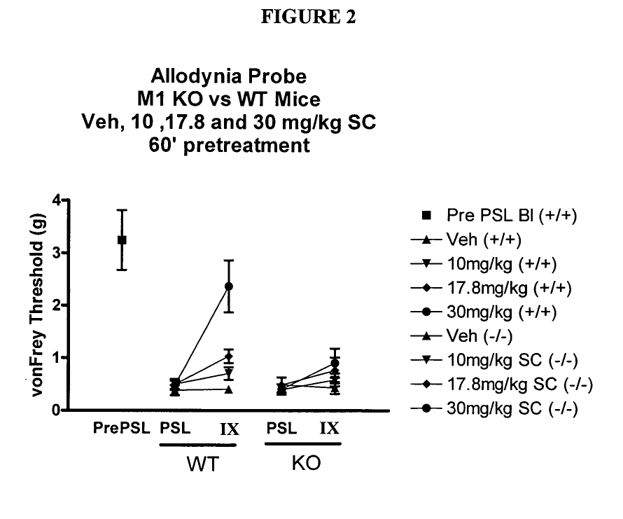

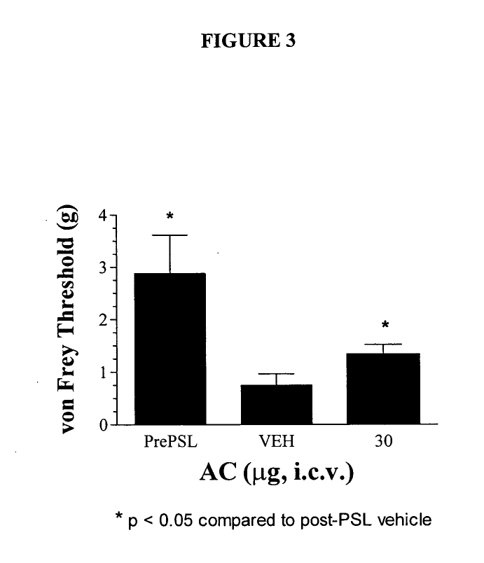

Further, as depicted in FIG. 2, the compound of Formula IX significantly reversed tactile allodynia in mice with PSL neuropathic injury after intracerebroventricular (i.c.v.) administration, suggesting a supraspinal mechanism of action consistent with M(1) receptor distribution.

### References

The following references are hereby incorporated by reference in their entirety:

Bartolini A., Ghelardini C., Fantetti L., Malcangio M., Malmberg-Aiello P., Giotti A. Role of muscarinic receptor subtypes in central antinociception. Br. J. Pharmacol. 105:77-82, 1992.

Brodie M. S. and Proudfit H. K. Hypoalgesia induced by the local injection of carbachol into the nucleus raphe magnus. Brain Research 291:337-342, 1984.

Capone F., Aloisi A. M., Carli G., Sacerdote P., Pavone F. Oxotremorine-induced modifications of the behavioral and neuroendocrine responses to formalin pain in male rats. Brain Res. 830:292-300, 1999.

Duttaroy A, Gomeza J, Gan J W, Siddiqui N, Basile A S, Harman W D, Smith P L, Felder C C, Levey A I, Wess J. Evaluation of muscarinic agonist-induced analgesia in muscarinic acetylcholine receptor knockout mice. Mol. Pharmacol. 62:1084-93, 2002.

Hartvig P., Gillberg P. G., Gordh T. Jr., Post C. Cholinergic mechanisms in pain and analgesia. Trends Pharmacol. Sci. Dec. Suppl.:75-79, 1989.

Hwang J.-H., Hwang K.-S., Leem J.-K., Park P.-H., Han S.-M., Lee D.-M. The antiallodynic effects of intrathecal cholinesterase inhibitors in a rat model of neuropathic pain. Anesthesiology 90:492-494, 1999.

Lee E. J., Sim J. Y, Park J. Y., Hwang J. H., Park P. H., Han S. M. Intrathecal carbachol and clonidine produce a synergistic antiallodynic effect in rats with a nerve ligation injury. Can J Anaesth 49:178-84, 2002.

Naguib M. and Yaksh T. L. Characterization of muscarinic receptor subtypes that mediate antinociception in the rat spinal cord. Anesth. Analg. 85:847-853, 1997.

Pedigo N. W., Dewey W. L. and Harris L. S. Determination and characterization of the antinociceptive avtivity if intraventricularly administered acetylcholine in mice. J. Pharmacol. Exp. Ther. 193: 845-852, 1975.

Prezewlocka B., Mika J., Capone F., Machelska H., Pavone F. Intrathecal oxotremorine affects formalin-induced behavior and spinal nitric oxide synthase immunoreactivity in rats. Pharmacol. Biochem. Behav. 62:531-536, 1999.

Shannon H. E., Womer D. E., Bymaster F. P., Calligaro D. O., DeLapp N. C., Mitch C. H., Ward J. S., Whitesitt C. A., Swedberg M. D. B., Sheardown M. J., Fink-Jensen A., Olesen P. H., Rimvall K., Sauerberg P. In vivo pharmacology of butylthio[2.2.2.] (LY297802/NNC11-1053), an orally acting antinociceptive muscarinic agonist. Life Sci. 60:969-976, 1997.

Sheardown M. J., Shannon H. E., Swedberg M. D. B., Suzdak P. D., Bymaster F. P., Olesen P. H., Mitch C. H., Ward J. S., Sauerberg P. M1 receptor agonist activity is not a requirement for muscarinic antinociception. J. Pharmacol. Exp. Ther. 281:868-875, 1997.

1. A method for treating neuropathic pain comprising:
identifying a subject in need of such treatment; and
providing the subject with an effective amount of at least one compound that selectively activates the M(1) receptor subtype, whereby one or more symptoms of the neuropathic pain are reduced.

2. The method of claim 1, wherein the subject presents hyperalgesia.

3. The method of claim 1, wherein the subject presents allodynia.

4. The method of claim 1, wherein the neuropathic pain is associated with diabetes, viral infection, irritable bowel syndrome, amputation, cancer, or chemical injury.

5. The method of claim 1, wherein the at least one compound that selectively activates the M(1) receptor subtype does not alleviate acute pain.

6. The method of claim 1, wherein the compound is selected from the group consisting of the compounds of Formulas VII, VIII, and IX:

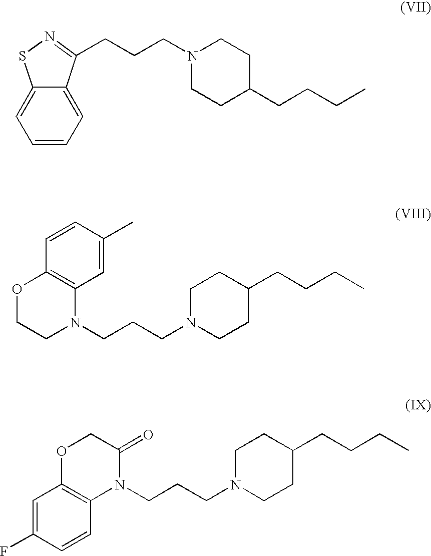

7. A method of identifying a compound that alleviates hyperalgesia or allodynia in a subject, comprising:
providing the subject with at least one muscarinic receptor test compound; and
determining if the at least one test compound reduces hyperalgesia or allodynia in the subject.

8. The method of claim 7, wherein the at least one test compound is selective for the M(1) or M(4) but not M(2) or M(3) receptor.

9. The method of claim 7, wherein the at least one test compound is selective for the M(1) receptor.

10. The method of claim 7, wherein the hyperalgesia is thermal hyperalgesia.

11. The method of claim 7, wherein the allodynia is tactile allodynia.

12. A pharmaceutical composition comprising an effective amount of at least one compound that selectively activates the M(1) receptor subtype in an amount effective to reduce one or more symptoms of neuropathic pain.

13. The composition of claim 12, wherein the compound is selected from the group consisting of the compounds of Formulas VII, VIII, and IX: [chemical structure]
